(19) United States
(12) Patent Application Publication
(10) Pub. No.: US 2023/0224293 A1
(43) Pub. Date: Jul. 13, 2023

(54) MEDICAL DEVICE COMMUNICATION CERTIFICATE MANAGEMENT

(71) Applicant: ICU Medical, Inc., San Clemente, CA (US)

(72) Inventors: Mark C. Rohlwing, Mesa, AZ (US); S. Sree Vivek, Chennai (IN); Hrishikesh Anil Dandekar, Pune (IN); Dharani Kumar Srinivasan, Chennai (IN); Rahul K R, Chennai (IN); Vasile Bora, San Diego, CA (US); Satyendra Singh Jadaun, Kota (IN)

(21) Appl. No.: 18/187,503

(22) Filed: Mar. 21, 2023

Related U.S. Application Data
(63) Continuation of application No. PCT/US2022/076417, filed on Sep. 14, 2022.

(30) Foreign Application Priority Data
Sep. 17, 2021 (IN) 202111042233

(51) Int. Cl. H04L 9/40; H04L 9/08

(52) U.S. Cl. CPC H04L 63/0823; H04L 63/0876; H04L 9/0825

(57) ABSTRACT

Techniques for managing secure communication certificates for medical devices in a clinical environment are provided. A short-lived, limited-use token may be uniquely assigned to a medical device. The medical device can self-provision a secret key and corresponding public key based on a unique identifier in the token. The medical device generates a certificate signing request (“CSR”) that includes the public key, and sends the CSR and the token to a verification system that serves as an intermediary between medical devices and a certificate authority. The intermediary may only send the CSR to the certificate authority (“CA”) for a certificate if the intermediary is able to validate the token.

### CROSS-REFERENCE TO RELATED APPLICATIONS

All applications for which a foreign or domestic priority claim is identified in the Application Data Sheet as filed with the present application are hereby incorporated by reference and made part of this specification.

### TECHNICAL FIELD

This disclosure relates to the field of medical device management, and particularly to systems and methods for secure communication with medical devices.

### BACKGROUND

Electronic medical devices often have processors and other computing components. Such medical devices may execute software and communicate with other computing systems via a network. Secure network communication may involve the establishing secure connections based on the identities of the devices participating in the communication, and encryption and decryption of data transmitted via the secure connections.

### SUMMARY

Various techniques for managing secure communication certificates for medical devices in a clinical environment are described herein. These techniques may include uniquely assigning a short-lived, single-use token to a medical device (e.g., an infusion pump) that is being configured for network access within a clinical environment. The token may be bound to the medical device by including a fingerprint of the medical device's public key in the token. For example, the medical device can self-provision a secret key and corresponding public key such that the secret key is never exposed outside the medical device. The medical device may provide a key fingerprint (e.g., a hashed version of the medical device's public key) to a setup device that generates the token and provides the token to the medical device. The medical device generates a certificate signing request (“CSR”) that includes an identifier of the medical device (e.g., received via the token) and medical device's public key. The public key (and optionally some additional data, such as the identifier) may also be signed using the medical device's secret key, and the signature may be included in the CSR to prove possession of the secret key that corresponds to the public key. The medical device sends the CSR and the token to a verification system that serves as an intermediary between medical devices and a certificate authority. The intermediary may only send the CSR to the certificate authority (“CA”) for a certificate if the intermediary is able to verify the signature in the token, verify that a fingerprint of the public key in the CSR matches the key fingerprint in the token, verify that the token has not expired, verify that the token has not been previously used, and verify that the token is assigned to the medical device making the request. Once the CA generates the certificate, the certificate is provided to the medical device and used by the medical device in secure communications with a hospital medication safety system and/or other devices on a network in the clinical environment. These and other embodiments are described in greater detail below with reference to FIGS. 1-6. Although many of the examples are described in the context of medical devices, functions, and environments (including infusion pumps, medication dispensing functions, and hospital or clinical environments), the techniques described herein can be applied to other types of devices, functions, and environments.

### BRIEF DESCRIPTION OF DRAWINGS

Throughout the drawings, reference numbers may be re-used to indicate correspondence between referenced elements. The drawings are provided to illustrate example embodiments described herein and are not intended to limit the scope of the disclosure.

FIG. 1 is a block diagram of an example medical device, setup device, verification system, and certificate authority in a hybrid network architecture according to some embodiments.

FIG. 2 is a block diagram illustrating data flows and interactions during a medical device setup and cryptographic key pair generation procedure according to some embodiments.

FIG. 3 is a diagram illustrating data flows and interactions during a certificate request and generation procedure according to some embodiments.

FIG. 4 is a flow diagram of an illustrative routine performed by a verification system to verify a certificate signing request according to some embodiments.

FIG. 5 is a block diagram illustrating data flows and interactions for certificate revocation list distribution according to some embodiments.

FIG. 6 is a diagram illustrating data flows and interactions during a rekey and recertification procedure according to some embodiments.

### DETAILED DESCRIPTION OF ILLUSTRATIVE EMBODIMENTS

The present disclosure is directed to management of cryptographic keys (also referred to simply as “keys”) and secure communication certificates (also referred to simply as “certificates”) for medical devices in a clinical environment.

Some existing methods of key generation involve generating a secret key outside of a device and providing the secret key to the device for use. For example, the secret key may be generated by a manufacturer and then saved into memory of the device at the time of manufacture or sale. As another example, the secret key may be obtained when the device boots up, such as by connecting to a key server or other provider of secret keys. However, these methods expose the secret key outside of the device and therefore pose a security risk. Some existing methods of identity and certificate management involve signing a cryptographic key to generate a certificate, and then providing the certificate to multiple medical devices. However, this process results in using the same certificate and therefore identity for each medical device. Moreover, this process risks the security of each of the medical devices if the cryptographic key becomes compromised, and makes revoking a certificate difficult or impractical.

Some aspects of the present disclosure address the issues noted above, among others, through the use of a short-lived, limited-use token that is uniquely assigned to a medical device. When a medical device, such as an infusion pump, is deployed in a clinical environment, there may be an initial configuration process to get the medical device connected to the communication network within the clinical environment. In some embodiments, a secure setup device may communicate with the medical device via a wired or direct short-range wireless connection. The setup device may generate a token for the medical device, and the token may include identity data that provides an identity of the device. The token may be uniquely-assigned to the medical device in the sense that the identity data that provides the identity of the device is unique to that medical device (e.g., a unique hardware identifier). In some embodiments, the token may include a key fingerprint, such as a hashed version of the medical device's public key, received from the medical device. The token may be short-lived in the sense that there is a relatively short duration of time before expiration of the token, or a specific expiration date/time is assigned to the token when it is issued. The token may be limited-use in the sense that the token may only be used a limited number of times (e.g., once) to obtain a certificate. In some embodiments, the token may include parameters in addition to the identity data to track the usage and time-bound properties of the token. For example, the token may be a multi-field data structure that includes an expiration parameter that specifies a date and/or time at which the token will expire. In order to ensure that the identity, expiration, and other parameters of the token are not tampered with, the token may be a cryptographically signed token. For example, the token may be signed by the token issuer using a secret key of the issuer. The signature can be verified, and the token's contents confirmed as being unaltered, using the corresponding public key of the issuer.

Additional aspects of the present disclosure relate to self-generating cryptographic keys for secure communication. A medical device can generate the secret key and corresponding public key that the medical device will use for secure communication. The secret key can be stored in a secure memory area of the medical device from where it can be accessed only for internal cryptographic operations and that is otherwise not accessible outside of the medical device. Thus, because the secret key is generated within the medical device and stored in a secure memory area of the medical device, the secret key is not exposed to potential security risks involved with generation of the secret key outside of the device (e.g., by a key server), transmission of the secret key over a network (e.g., from the key server), etc.

Further aspects of the present disclosure relate to secure management of certificate signing requests using a verification system that serves as an intermediary between the medical device and the certificate authority. In some embodiments, the verification system can be on a local network of the clinical environment, and may be the only system or one of a limited set of systems that communicates outside of the local network. This configuration can ensure that all medical devices communicate with the verification system for cryptographic needs and minimizes exposure of the medical devices outside of the clinical environment. When a medical device generates a request such as a certificate signing request (“CSR”) to obtain a certificate, the request can be sent to the verification system rather than directly to the certificate authority (“CA”) that will generate the certificate. The verification system can verify the CSR prior to sending the CSR to the CA. For example, the medical device may provide, in addition to the CSR, the signed token that the medical device received previously. The verification system can use the token to verify that the CSR is being received from the proper medical device (e.g., based on the setup device signature in the token, a key fingerprint in the token, etc.), that the CSR is being made within a required period of time (e.g., based on an expiration parameter in the token), and that the token has not been used previously to make a CSR request (e.g., based on data maintained at the verification system). If the CSR and associated token satisfy the verification criteria, the verification system can send the CSR to the CA. The CA can then generate the certificate.

In some embodiments, the medical device's identity data is present in the CSR, and the verification system verifies whether the identity is the same as the identity present in the token. If so, the verification system sends the CSR to CA, and the CA uses the device identity in the CSR to issue a certificate. In some embodiments, there will be a subject field in the certificate that needs more information than what is present in the token and/or CSR. For example, a hospital name and location may be added, a customer identification number may be added, etc. This information can be provided by the verification system and/or the CA may have access to this information.

Additional aspects of the present disclosure relate to use of a hybrid network architecture to implement the certificate generation and management process. In some embodiments, the hybrid network architecture includes a network for devices within the clinical environment (e.g., a local area network or wide area network of a hospital) and another network for the CA (e.g., a separate local area network or wide area network of a cloud provider). For example, the CA may be implemented by a cloud computing provider and accessible to clinical environment network via the internet. In such a hybrid network architecture, the verification system implemented within the clinical environment can provide a single point for access and communicating with the CA, thereby reducing or eliminating the need for multiple medical devices (dozens, hundreds, or more) to each establish their own network connections to the CA. Instead, the medical devices can each communicate with the verification system (e.g., to send CSRs, receive certificates, verify signatures, etc.), and the verification system can maintain a connection to the CA. This can reduce the attack surface exposed outside of the clinical environment, and facilitate the use of a single connection to the CA that may provide better performance than may otherwise be available to the individual medical devices.

Further aspects of the present disclosure relate to management of certificate revocation lists by the verification system. A certificate revocation list (“CRL”) is a list of certificates, previously generated by a CA, that have been revoked before their scheduled expiration dates. When one device receives a certificate from another device while establishing a secure communication connection, the CRL can be checked to see whether the certificate has been revoked and the connection should therefore not be established. Certificates may be revoked for any of a variety of reasons, such as a compromised secret key, a compromise of the issuing CA, a replacement certificate being issued (e.g., a re-certification), other security vulnerabilities, etc. Typically, devices obtain CRLs directly from a CA, such as in response to a CRL request. This can be inefficient and cause undesirable traffic to and from the CA. When a hybrid network architecture is implemented as described herein, the verification system can prefetch and maintain an up-to-date CRL from the CA. The CRL can be pushed to medical devices in the clinical environment network (or the medical devices can request the CRL from the verification system) instead of the medical devices requesting the CRL from the CA. Use of the verification system in the hybrid network architecture in this manner can both ensure that the medical devices have the most up-to-date CRLs, and also reduce traffic to the CA to obtain the CRLs. In some embodiments, the verification system may obtain or generate a delta CRL that includes only revocation details that have been changed since the last delta CRL was obtained/generated or since the last full CRL was obtained. Periodically obtaining, generating, and/or distributing delta CRLs instead of full CRLs can further reduce the time and bandwidth in comparison with the full CRLs.

Various aspects of the disclosure will now be described with regard to certain examples and embodiments, which are intended to illustrate but not limit the disclosure. Although aspects of some embodiments described in the disclosure will focus, for the purpose of illustration, on particular examples of medical devices, cryptographic key generation algorithms, and the like, the examples are illustrative only and are not intended to be limiting. In some embodiments, the systems and methods described herein may be applied to additional or alternative medical devices, cryptographic algorithms, etc.

### Overview of Example Network Environment

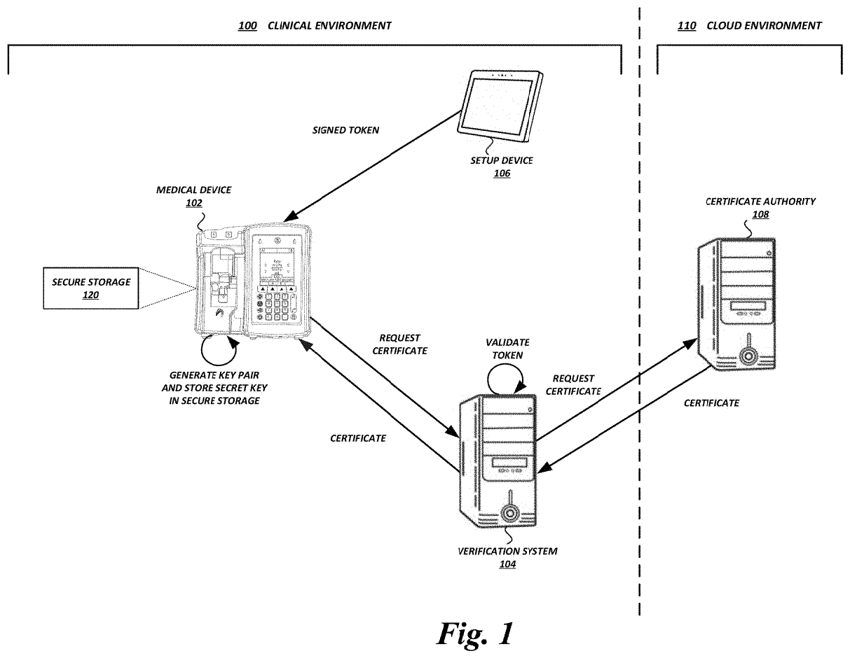

FIG. 1 shows a clinical environment 100 and a cloud environment 110 in which aspects of the present disclosure may be implemented for generating and managing cryptographic keys and corresponding certificates for medical devices. In some embodiments, the clinical environment 100 and the cloud environment 110 may each be implemented on one or more wired and/or wireless private networks, such as local area networks (“LANs”), virtual local area networks (“VLANs”), wide area networks (“WANs”), etc. The clinical environment 100 and the cloud environment 110 (or individual devices thereof) may communicate with each other via one or more wired and/or wireless networks. For example, the clinical environment 100 and cloud environment 110 may communicate via a publicly-accessible network of linked networks, possibly operated by various distinct parties, such as the Internet. In some cases, the clinical environment 100 and cloud environment 110 may communicate via a private network, local area network, virtual local area network, wide area network, global area network, cable network, satellite network, cellular data network, etc., or a combination thereof, some or all of which may or may not have access to and/or from the Internet.

A cloud environment 110 may be a cloud-based platform configured to communicate with multiple clinical environments. The cloud environment 110 may include a collection of services, which are delivered via the network as web services. For example, the cloud environment may include a certificate authority (“CA”) 108 that generates and manages certificates for proof of identity and secure communication. The CA 108 may be implemented on one or more server computing devices with processors and memory.

The CA 108 may be implemented using various logical and/or physical architectures. In some embodiments, the CA 108 may be implemented as an isolated CA for a single clinical environment 100. For example, the CA 108 may include a root CA and one or more subordinate CAs or intermediate CAs that collectively generate and manage certificates for a single clinical environment 100, and are physically and/or logically isolated from the root CA and sub CAs for other clinical environments. In some embodiments, the CA 108 may be implemented with a root CA that is shared across multiple clinical environments. For example, the CA 108 may include a single root CA for all clinical environments (or a subset thereof), and separate isolated sub CAs for each separate clinical environment (or subsets thereof). In some embodiments, the CA 108 may be implemented with a root CA and sub CAs that are shared across multiple clinical environments. For example, the CA 108 may include a single root CA and a set of sub CAs, and each of the sub CAs can generate and manage certificates for each of multiple clinical environments.

A clinical environment 100 may include one or more healthcare facilities (e.g., hospitals), each of which may include various devices and/or systems. In some embodiments, the clinical environment 100 may include any number of medical devices 102, verification systems 104, and setup devices 106.

A medical device may 102 may be any electronic medical device, or medical device with an electronic component, configured to communicate with other devices over a communication network. In some embodiments, a medical device 102 may be an infusion pump.

The setup device 106 may be any electronic device configured to electronically communicate with other devices. In some embodiments, the setup device 106 may be any handheld or substantially portable electronic device with a processor, memory, communication interface, and user interface. For example, the setup device 106 may be a smart phone, personal digital assistant, tablet, laptop computer, desktop computer with a mobile base, etc. The setup device 106 may allow user interaction to initiate setup processes in which the setup device 106 generates signed cryptographic tokens, and provides the cryptographic tokens and other data, such as network connectivity and configuration data, to medical devices 102 as described in greater detail below.

The verification system 104 may be any electronic device configured to communicate with other devices. In some embodiments, the verification system 104 may be a desktop computer, server computer, network appliance, or the like. The verification system 104 can serve as a gateway for some or all devices and systems of the clinical environment 100 to communicate with the cloud environment. For example, the verification system 104 may send CSRs on behalf of medical devices 102 to the CA 108 in the cloud environment 110, send certificates from the CA 108 to medical devices 102, manage acquisition and distribution of CRLs, etc.

In some embodiments, the clinical environment 100 may also or alternatively include one or more clinical IT systems (not shown). For example, the clinical environment may include a hospital information system (“HIS”) designed to manage the facilities' operation, such as medical, administrative, financial, and legal issues and the corresponding processing of services. The HIS can include one or more electronic medical record (“EMR”) or electronic health record (“EHR”) systems.

The example devices and systems of the clinical environment 100 and cloud environment 110 shown in FIG. 1 and described herein are illustrative only, and are not intended to be limiting, required, or exhaustive. In some embodiments, a clinical environment 100 and/or cloud environment 110 may include additional, alternative, and/or fewer devices and/or systems. For example, although only one instance of a medical device 102, verification system 104, and setup device 106 are shown in FIG. 1, in practice any number or combination of devices and systems may be included in a clinical environment 100. A single clinical environment 100 may have dozens, hundreds, or more individual medical devices 102, and the medical devices 102 may be the same as or different than each other.

In some embodiments, as shown, various devices and systems may participate in the process of key and certificate generation. A setup device 106 can generate a cryptographically signed token with identity information for the medical device 102, and provide the token to the medical device 102. The medical device 102 can generate a secret key and corresponding public key, and then generate a CSR. A verification system 104 can validate the token and send the CSR to a CA 108. The CA 108 can sign the key included in the CSR and send back the signed key in a certificate. Example routines and implementations of key and certificate generation and management are described in greater detail below.

### Identity Assignment and Key Generation

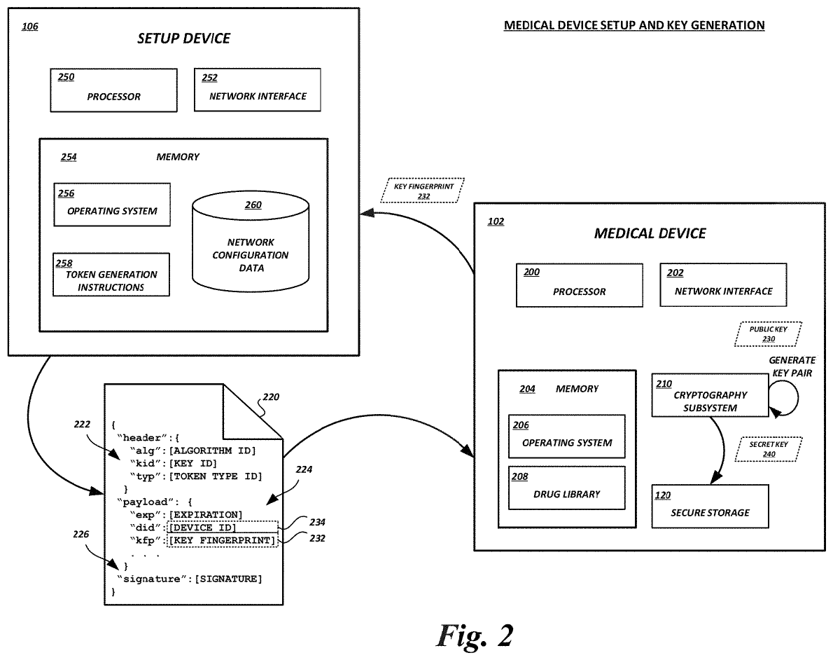

FIG. 2 illustrates example interactions between a medical device 102 and a setup device 106 during setup of the medical device 102.

In some embodiments, as shown, the medical device 102 may include: one or more computer processors 200, such as physical central processing units (“CPUs”); one or more network interfaces 202, such as network interface cards (“NICs”); and a computer readable memory 204, such as random access memory (“RAM”) and/or other non-transitory computer-readable media. The computer readable memory 204 may include computer program instructions that the processor 200 executes in order to implement one or more embodiments. For example, the computer readable memory 204 can store an operating system 206 that provides computer program instructions for use by the computer processor 200 in the general administration and operation of the medical device 102. The computer readable memory 204 may also include a drug library 208 with data regarding medications that may be administered using the medical device 102.

The medical device 102 may also include a cryptography subsystem 210 for performing cryptographic operations (e.g., key generation, encryption, decryption, etc.), and a secure storage 120 to store a secret key generated by the cryptography subsystem 210. The cryptography subsystem 210 may be implemented in hardware, or as a combination of software and hardware to execute the software. The secure storage 120 may be a non-volatile secure element that stores one or more keys persistently, even after the medical device 102 is powered off. Although the cryptography subsystem 210 and secure storage 120 are shown in FIG. 2 as being physically separate from each other and from other components of the medical device 102, in some embodiments one or both of the cryptography subsystem 210 and/or secure storage 120 may be implemented as a subcomponent of another component.

In some embodiments, the medical device 102 may be or include an infusion pump with various components to perform infusion pump operations. For example, an infusion pump may include a motor controller unit (“MCU”) configured to control a motor (not shown) that dispenses medication from a medication container based on instructions received via the clinical environment network and/or via a user interface of the medical device 102.

In some embodiments, as shown, the setup device 106 may include: one or more computer processors 250, such as physical CPUs; one or more network interfaces 252, such as NICs; and a computer readable memory 254, such as RAM and/or other non-transitory computer-readable media. The computer readable memory 254 may include computer program instructions that the computer processor 250 executes in order to implement one or more embodiments. For example, the computer readable memory 254 can store an operating system 256 that provides computer program instructions for use by the computer processor 250 in the general administration and operation of the setup device 106. The computer readable memory 254 may also include token generation instructions 258 for generating signed tokens used in key and certificate generation as described herein.

In some embodiments, the computer readable memory 254 may also include network configuration data 260 for use in configuring medical devices 102 to access a network of the clinical environment 100. For example, when a medical device 102 is manufactured, it may be capable of communication over networks (e.g., it may include a network interface and related software), but may not be configured for accessing any specific network. In order to access a specific network when deployed within a clinical environment 100, the medical device 102 may require various configuration settings (e.g., network name, network addresses, etc.). The network configuration data 260 may include such configuration settings, or data from which the configuration settings can be derived.

The cryptography subsystem 210 or some other module or component of the medical device 102 can generate one or more cryptographic keys. In the illustrated example, the medical device 102 generates a secret key 240 and stores the secret key 240 in secure storage 120. By generating the secret key 240 within the medical device 102, exposure of the secret key 240 to other devices can be prevented potentially indefinitely.

In addition to generating a secret key 240, the cryptography subsystem 210 or some other module or component of the medical device 102 can generate a corresponding public key 230 to be signed by a CA and used in secure communications. For example, the cryptography subsystem 210 may generate a key pair for use in asymmetric cryptography, such as public key encryption. The public key 230 may correspond to the secret key 240 in the sense that the public key 230 can be used to decrypt data encrypted with the secret key 240, and/or the secret key 240 can be used to decrypt data encrypted with the public key 230. Thus, the medical device 102 may cryptographically sign data by encrypting the data using the secret key 240 and include the encrypted version with the original data. In some embodiments, the signing procedure may involve hashing the data and encrypting the hash. The resulting encrypted hash may be used as the signature, and may be sent with the original un-hashed and un-encrypted data. Recipients can use the medical device's corresponding public key 230 to decrypt the encrypted version (e.g., to derive the hash from the signature) and verify that the original data has not been altered (e.g., by checking whether the hash matches a hash of the original data). In addition, the medical device's public key 230 may be provided to or otherwise obtained by other devices in a certificate that has been signed by a CA, thereby verifying the source of the data as the medical device 102.

To configure the medical device 102, the setup device 106 may establish a wired or wireless connection with the medical device 102. For example, the setup device 106 may use near-field wireless communication such as Bluetooth, Wi-Fi Direct, Near-Field Communication (“NFC”), or the like to communicate directly with the medical device 102. As another example, the setup device 106 may be temporarily coupled to the medical device 102 using a network cable, a universal serial bus (“USB”) cable, or the like. With a connection to the medical device 102 established, the setup device 106 may send network configuration data (settings, credentials, etc.) to the medical device 102.

In some embodiments, the medical device 102 may provide information to the setup device 106 during the setup process. For example, the cryptography subsystem 210 may generate a key fingerprint 232 of the public key 230, and provide the key fingerprint 232 to the setup device 106. The key fingerprint 232 may be an encoded version of the public key 230 (and, in some cases, additional information). The cryptography subsystem 210 may generate a hash of the public key 230, and the hash may be provided to the setup device 106 as the key fingerprint 232 for use in the setup process, as described below.

In addition to (or instead of) providing network configuration data to the medical device 102, the setup device 106 can provide identity data that the medical device 102 may use to obtain a certificate. The identity data may include a unique device ID that is assigned to the medical device 102. For example, the identity data may be a numeric or alphanumeric string generated using an algorithm that produces unique or substantially unique data (e.g., based on a pseudo-random number generator, a hash function, other functions, or some combination thereof). As another example, the identity data may be selected from a listing of unique identifiers to be assigned to medical devices.

In some embodiments, the identity data may be provided in a cryptographically signed data token or other data structure that may or may not include other data items. For example, the identity data may be provided in a JavaScript Object Notation (“JSON”) file, a JSON Web Token (“JWT”), an eXtensible Markup Language (“XML”) file, or some other structured data object.

FIG. 2 shows an example signed token 220. The token 220 includes a header 222 with fields for metadata items, including a data item specifying the algorithm with which the token has been signed (in the “alg” field), a data item specifying an identifier of a public key used to sign the token (in the “kid” field), and a data item specifying the type of signed token being presented (in the “typ” field). The token 220 also includes a payload 224 with fields for payload data items, including a data item specifying when the token 220 expires (in the “exp” field), a data item specifying the device ID 234 assigned to the medical device (in the “did” field), and a data item for the key fingerprint 232 of the medical device's public key (in the “kfp” field). The token 220 also includes a signature 226 with a field for the signature that can be used to validate the token 220. The sections and fields shown in FIG. 2 and described herein are illustrative only, and are not intended to be limiting, required, or exhaustive. In some embodiments, additional, alternative, or fewer sections and/or fields may be included in the token 220. For example, the payload 224 may include one or more fields for network configuration data that the medical device 102 is to use to join the network of the clinical environment 100.

### Certificate Generation

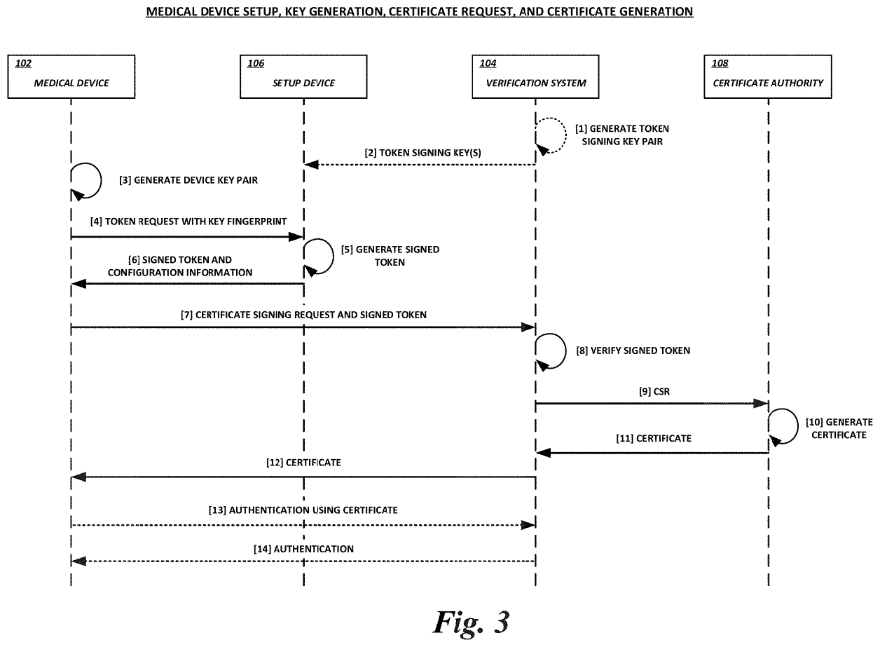

FIG. 3 illustrates example interactions between a medical device 102, setup device 106, verification system 104, and CA 108 during the process of enrolling a setup device 106 with the verification system 104 to generate signed tokens, setting up a medical device 102, generating keys, requesting certificates, and obtaining certificates according to some embodiments.

At [1] the verification system 104 may generate a key pair for use by the setup device 106 in generating tokens. For example, the setup device 106 may participate in an enrollment procedure with the verification system 104. Advantageously, the setup device 106 is a substantially portable device capable of establishing direct wired or wireless connections with medical devices 102 that have not yet been configured to connect to the network of the clinical environment 100 and would therefore be unable to communicate with the verification system 104. Thus, the setup device 106 is enrolled with the verification system 104 so that the setup device 106 can be transported through the clinical environment 100 and set up medical devices 102 on behalf of the verification system 104. In some embodiments, the enrollment procedure may be performed once per setup device 106, and an enrolled setup device 106 may then generate signed tokens for multiple medical devices 102.

During the enrollment procedure, the setup device 106 obtains a public key and corresponding secret key at [2]. The verification system 104 may generate the key pair so that tokens generated by the setup device 106 can be signed and the verification system 104 (and other systems or devices) can verify that the contents of the tokens have not been altered and the source of the tokens is the setup device 106. In some embodiments, the setup device 106 may generate the key pair or otherwise obtain the key pair from a source other than the verification system 104. In this case, the setup device 106 can provide its public key (e.g., in a certificate signed by a CA) to the verification system 104 during the enrollment procedure.

At [3] the medical device 102 can generate a pair of cryptographic keys. For example, the medical device 102 may use a seed or other parameter in a key generation algorithm to generate a device-specific key pair. The secret key may be stored in a secure storage that is not accessible from outside the medical device 102, and therefore the secret key is never exposed outside of the medical device 102.

At [4], the medical device 102 can request a token and configuration data from the setup device 106. The request may include the key fingerprint of the public key of the medical device. In some embodiments, the setup device 106 may initiate the setup process with the medical device 102 rather than the medical device 102 sending a request to the setup device 106.

At [5] the setup device 106 can begin the process of configuring a medical device 102 for secure network communications. The setup device 106 may generate a token, and sign the token using the setup device's secret key. As shown in FIG. 2 and described above, the token 220 may be a multi-field data structure that includes a device ID, key fingerprint, expiration data, a signature, and/or various other data use during the setup process. In some embodiments, the expiration data may be generated to limit the valid life of the token to a relatively short period of time (e.g., x seconds, minutes, or hours) during which the medical device 102 is expected to be able to perform key generation and certificate request processing as described in greater detail below. The setup device 106 can provide the signed token and, in some cases, separate network configuration information, to the medical device 102 at [6]. The medical device 102 may then establish a connection to the network of the clinical environment 100 using network configuration information received from the setup device 106.

At [7] the medical device 102 may send a CSR to the verification system 104. The CSR may include or otherwise be associated with the public key 230 generated by the medical device 102, the device ID 234 assigned to the medical device 102, and/or various other data. The public key 230 (and optionally some additional data, such as the device ID 234) may also be signed using the medical device's 102 secret key 240, and the signature may be included in the CSR to prove possession of the secret key 240 that corresponds to the public key 230. In addition to sending the CSR, the medical device 102 may send the signed token that was received from the setup device 106 and from which the key pair was generated. The signed token may be provided with the CSR so that the verification system 104 can verify the identity of the medical device 102 and perform other request validation operations, as described in greater detail below.

At [8] the verification system 104 can verify the CSR and signed token. The signed token may be a short-lived, limited use token that is uniquely assigned to a particular medical device 102. If the token cannot be verified as being signed by the setup device 106, or if the signed token has expired, has been previously used, or is not assigned to the same device from which the CSR is received, the verification system 104 may not obtain a certificate for the medical device.

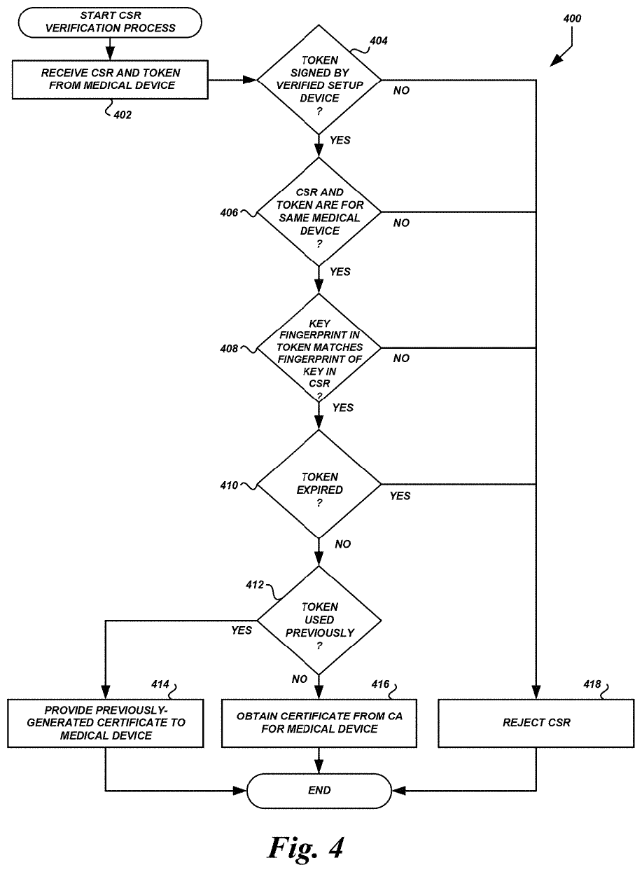

FIG. 4 illustrates a routine 400 that may be performed to verify the CSR and signed token. When the routine 400 is initiated, a set of executable program instructions stored on one or more non-transitory computer-readable media (e.g., hard drive, flash memory, removable media, etc.) may be loaded into memory (e.g., RAM) of a computing device and executed.

At block 402, the verification system 104 may receive a CSR and signed token from a medical device 102.

At decision block 404, the verification system 104 can evaluate the signature of the token. The signature can be evaluated to verify that setup device 106 has signed it, and to verify that the contents of the token have not been altered. For example, the verification system 104 can use the public key of the setup device 106 to verify whether the token has been signed by an enrolled setup device 106. If the token has not been signed by an enrolled setup device 106 and/or the contents of the token have been altered, the CSR may be rejected at block 418.

At decision block 406, the verification system 104 may verify that the device to which the token is uniquely assigned is the same device for which the CSR is being submitted. For example, the verification system 104 may check a device identifier in the CSR against a device identifier in the signed token to ensure they match. If the CSR cannot be verified as being for the same medical device 102 to which the signed token is assigned, the CSR may be rejected at block 418.

At decision block 408, the verification system 104 may verify that the fingerprint of the public key in the CSR matches the key fingerprint in the token. The verification system 104 may generate an encoded version of the public key in the CSR, which is purported to be the public key of the medical device 102 from which the CSR was received. For example, the verification system 104 may generate a hash of the public key in the CSR. The hash may be generated using the same hashing algorithm as was used by the medical device 102 to generate the key fingerprint in the token. The fingerprint generated from the public key in the CSR may then be compared to the key fingerprint from the token. If there is no match, then the CSR cannot be verified as being for the same medical device 102 to which the signed token is assigned, and the CSR may be rejected at block 418.

At decision block 410, the verification system 104 may verify whether the token has expired. The token may include expiration data that the verification system 104 can use to verify that the token has not expired. For example, the token may include an expiration date/time after which the token is expired. As another example, the token may be associated with a creation date/time, and a duration of time after the create date/time during which the token is valid. If the token has expired, the CSR may be rejected at block 418.

At decision block 412, the verification system 104 may determine whether the token has already been used to submit a CSR. For example, the verification system 104 may maintain a listing of tokens that have already been submitted with a CSR (e.g., token identifiers, copies of tokens, etc.). If the token has already been submitted with a CSR and a certificate has already been generated for the medical device 102, the verification system 104 may re-send the previously-generated certificate to the medical device 102 at block 414. In some embodiments, if the token has already been submitted with a CSR a threshold number of times (e.g., 1), the CSR may be rejected even if the token has not expired.

At block 416, if the verification system 104 can verify the token as being signed by an enrolled setup device 106, is assigned to the same medical device 102 as the CSR, includes a key fingerprint that matches the fingerprint of the key in the CSR, has not expired, and has not been previously used, then the verification system 104 may obtain a certificate for the medical device 102.

Returning to FIG. 3, at [9] the verification system 104 can send the CSR on to the certificate authority 108 for creation of a certificate.

At [10] the CA 108 can generate a certificate for the public key in the CSR. In some embodiments, the medical device's 102 identity data is present in the CSR, and the CA 108 uses the device identity in the CSR to issue a certificate. In some embodiments, there will be a subject field in the certificate that needs more information than what is present in the token and/or CSR. For example, a hospital name and location may be added, a customer identification number may be added, etc. This information can be provided by the verification system 104 to the CA 108 (e.g., via additional parameters in an API call to the CA 108) and/or the CA may otherwise have access to this information. The CA 108 may sign the certificate using its own secret key, and send the certificate to the verification system 104 at [11]. In some embodiments, the CA 108 may first send a notification (not shown) to the verification system 104 that the certificate has been generated. In response to the notification, the verification system 104 may request and receive the generated certificate from the CA 108.

At [12] the verification system 104 can send the certificate to the medical device 102 for use in secure communications. Subsequently, at [13] and [14], the medical device 102 may establish a secure connection with the verification system 104 (or some other device or system of the clinical environment 100) by engaging in a certificate-based authentication protocol using the certificate. For example, the medical device 102 may establish a Transport Layer Security (“TLS”) connection with the verification system 104 using the certificate. Such a connection may be used to receive medication administration data, operational instructions, sensitive data, software updates, and the like.

### Certificate Revocation

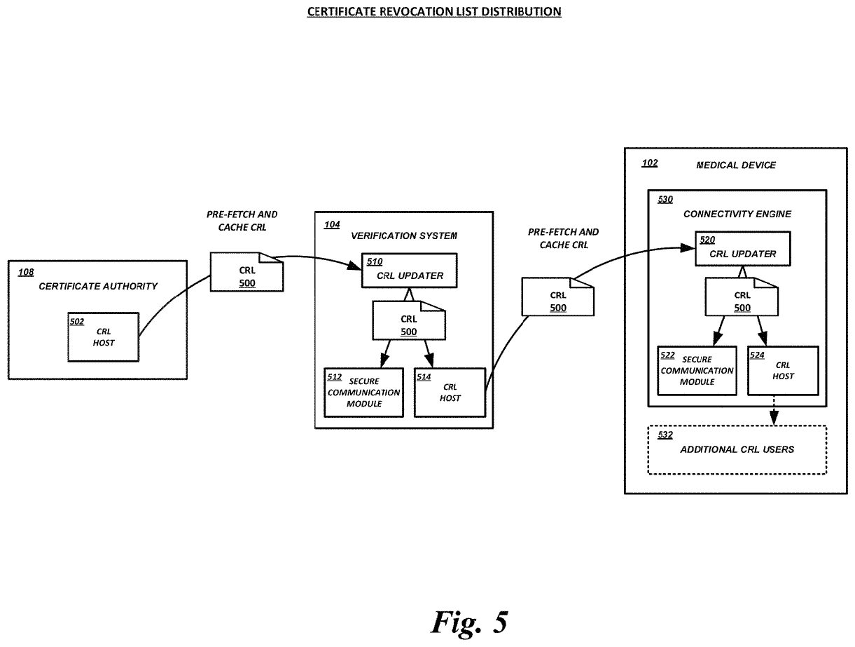

FIG. 5 illustrates example interactions between a medical device 102, verification system 104, and CA 108 to manage the distribution of certificate revocation lists (“CRLs”). A CRL is a list of certificates, previously generated by a CA, that have been revoked before their scheduled expiration dates. Certificates may be revoked for any of a variety of reasons, such as a compromised secret key, a compromise of the issuing CA, a replacement certificate being issued (e.g., a re-certification), other security vulnerabilities, etc. When one device receives a certificate from another device while establishing a secure communication connection, the CRL can be checked to see whether the certificate has been revoked and the connection should therefore not be established. Typically, devices obtain CRLs directly from a CA, such as in response to a CRL request. This can be inefficient and cause undesirable traffic to and from the CA. Moreover, when a hybrid network architecture is implemented in which a verification system 104 or some other system acts as an intermediary between medical devices 102 and a CA 108 as described herein, the medical devices 102 may not be able to directly access the CA 108.

In some embodiments, the verification system 104 can prefetch and maintain an up-to-date CRL from the CA 108. For example, the verification system 104 may prefetch a complete up-to-date CRL from the CA 108, or a delta CRL that includes only the changes from the last CRL or delta obtained by the verification system 104. By prefetching the CRL and caching it until it expires, the verification system 104 can save bandwidth and processing resources that would otherwise be expended to request and obtain the CRL during a secure connection protocol, such as a TLS handshake. Moreover, the verification system 104 can distribute or otherwise make the CRL available to other devices of the clinical environment 100 so that they may realize the same benefits.

As shown in FIG. 5, the CA 108 can include a CRL host 502 that maintains the CRL 500. At predetermined intervals or in response to events, a CRL updater 510 or some other component of the verification system 104 can obtain the CRL 500 from the CRL host 502 of the CA 108. The verification system 104 can store the CRL 500 in one or more locations. For example, the verification system 104 may include a secure communication module 512 that handles certificate and signature verification when the verification system 104 establishes secure connections with other devices and systems. The verification system 104 may store the CRL 500 in—or otherwise make the CRL 500 accessible to—the secure communication module 512 so that the secure communication module 512 can verify that the certificates with which it is presented have not been revoked. The verification system 104 may also store the CRL in a CRL host 514 that can make the CRL 500 accessible to other devices and systems of the clinical environment 100, such as medical devices 102.

A medical device 102 can prefetch and maintain an up-to-date CRL from the verification system 104. By prefetching the CRL and caching it until it expires, the medical device 102 can save bandwidth and processing resources during a secure connection protocol, as described above. In some embodiments, the CRL may be pushed to the medical device 102 by the verification system 104. In other embodiments, a CRL updater 520 or some other component of the medical device 102 can fetch the CRL 500 from the CRL host 514 of the verification system 104 at predetermined intervals or in response to various events. The medical device 102 can store the CRL 500 in one or more locations. For example, the medical device 102 may include a secure communication module 522 that handles certificate and signature verification when the medical device 102 establishes secure connections with other devices and systems. The medical device 102 may store the CRL 500 in—or otherwise make the CRL 500 accessible to—the secure communication module 522 so that the secure communication module 522 can verify that the certificates with which it is presented have not been revoked. The medical device 102 may also store the CRL in a CRL host 524 that can make the CRL 500 accessible to additional CRL users 532 in the medical device 102. For example, the medical device 102 may include physically or logically separate processing components, such as a connectivity engine 530 that manages the CRL and various connectivity operations, and a different component such as a user interface controller that manages various user interface operations. The various processing components may each initiate communication with other systems and devices (e.g., the verification system and/or a hospital medication safety system), perform digital signature verification, etc., and may therefore use the CRL 500.

With reference to an illustrative example, a medical device 102 may receive a drug library, or a new version of a drug library, from a hospital medication safety system (“HMSS”) or some other system. The drug library may be received with a digital signature generated by the HMSS, and a certificate of the HMSS. The medical device 102 may use the public key in the HMSS's certificate to verify the signature. In some embodiments, prior to verifying the signature, the medical device 102 may check the CRL 500 to determine whether the HMSS's certificate has been revoked. If it has been revoked, the medical device 102 may reject the drug library.

### Rekey and Recertification

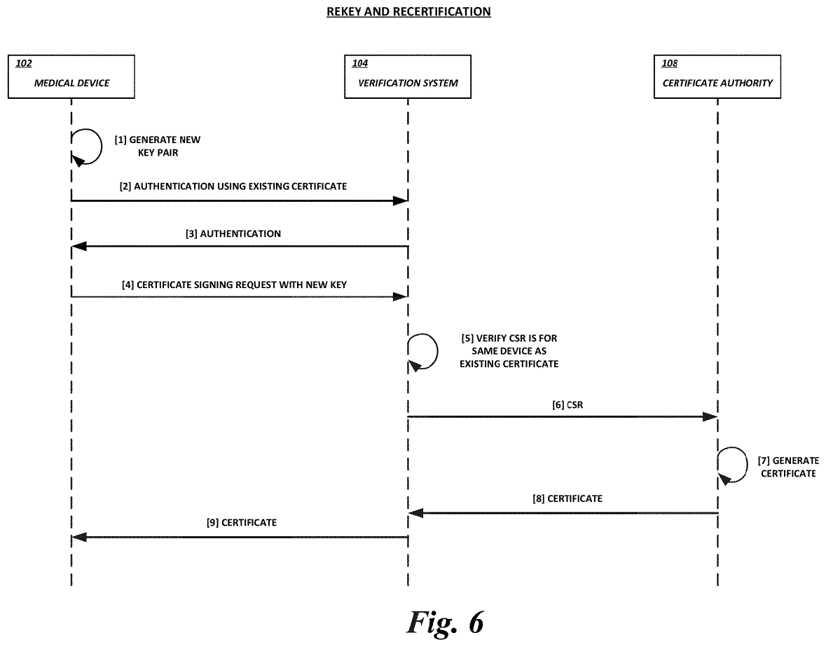

FIG. 6 illustrates example interactions between a medical device 102, verification system 104, and CA 108 to manage the rekeying of the medical device 102. Rekeying may be performed in response to detection or suspicion that a secret key of a medical device 102 has been compromised. In some embodiments, rekeying may be performed on a periodic basis to ensure that undiscovered security vulnerabilities do not persist for greater than a predetermined or dynamically determined period of time. In addition to generating new keys, the medical devices 102 also obtain new certificates for their public keys, as the existing certificates do not apply to the new keys that will be put into use. When the rekey and recertification procedure is performed before expiration of an existing certificate, the process may not require a new token, and therefore a setup process with a setup device 106 may not be needed.

At [1] the medical device 102 can generate a new pair of cryptographic keys to replace the existing pair of cryptographic keys currently in use. The medical device 102 may generate the keys based on the same key generation algorithm as in the key pair generation process described above, or the medical device 102 may use a different key generation algorithm and/or seed data. The new secret key may be stored in a secure storage that is not accessible from outside the medical device 102. Therefore, like the original (and any other previously-generated) secret key, the new secret key is never exposed outside of the medical device 102. The new secret key may replace the original or otherwise prior secret key, or the prior secret key may otherwise be deleted from the secure storage.

At [2] and [3], the medical device 102 may establish a secure connection with the verification system 104 by engaging in a certificate-based authentication protocol using the existing certificate. For example, the medical device 102 may establish a TLS connection with the verification system 104 using the existing certificate.

At [4] the medical device 102 may send a CSR to the verification system 104. The CSR may include the new public key generated by the medical device 102.

At [5] the verification system 104 can verify that the CSR is for the same medical device 102 as the device with which the secure connection has been established using the existing certificate. For example, the verification system 104 may check a device identifier in the CSR against a device identifier in the existing certificate to ensure they match. If the device identifiers match, the verification system 104 can send the CSR on to the certificate authority 108 at [6] for creation of a new certificate for the new key.

At [7] the CA 108 can generate a certificate for the public key in the CSR. The CA 108 may sign the certificate using its own secret key, and send the certificate to the verification system 104 at [8]. In some embodiments, the CA 108 may first send a notification (not shown) to the verification system 104 that the certificate has been generated. In response to the notification, the verification system 104 may request and receive the generated certificate from the CA 108. At [9] the verification system 104 can send the certificate to the medical device 102 for use in secure communications.

### Other Considerations

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

Many other variations than those described herein will be apparent from this disclosure. For example, depending on the embodiment, certain acts, events, or functions of any of the algorithms described herein can be performed in a different sequence, can be added, merged, or left out altogether (e.g., not all described acts or events are necessary for the practice of the algorithms). Moreover, in certain embodiments, acts or events can be performed concurrently, e.g., through multi-threaded processing, interrupt processing, or multiple processors or processor cores or on other parallel architectures, rather than sequentially. In addition, different tasks or processes can be performed by different machines and/or computing systems that can function together.

The various illustrative logical blocks, modules, and algorithm elements described in connection with the embodiments disclosed herein can be implemented as electronic hardware, computer software, or combinations of both. To clearly illustrate this interchangeability of hardware and software, various illustrative components, blocks, modules, and elements have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the embodiments disclosed herein can be implemented or performed by a machine, such as a computer processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A computer processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, or algorithm described in connection with the embodiments disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC. The ASIC can reside in a user terminal. In the alternative, the processor and the storage medium can reside as discrete components in a user terminal.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements, and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Unless otherwise explicitly stated, articles such as “a”, “an”, or “the” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B, and C” can include a first processor configured to carry out recitation A working in conjunction with a second processor configured to carry out recitations B and C.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, certain embodiments described herein can be implemented within a form that does not provide all of the features and benefits set forth herein, as some features can be used or practiced separately from others. All such modifications and variations are intended to be included herein within the scope of this disclosure. Further, additional embodiments created by combining any two or more features or techniques of one or more embodiments described herein are also intended to be included herein within the scope of this disclosure.

What is claimed is:

1. A system for managing communication certificates for medical devices, the system comprising:
a medical device comprising a secure memory; and
a verification system configured to serve as an intermediary between the medical device and a certificate authority;
wherein the medical device is configured to:
obtain a signed token comprising identity data and expiration data;
generate a secret cryptographic key and a corresponding public cryptographic key using the identity data;
store the secret cryptographic key in the secure memory;
generate a certificate signing request comprising the public cryptographic key; and
send the certificate signing request and the signed token to the verification system; and
wherein the verification system is configured to:
receive the certificate signing request and the signed token from the medical device;
verify a signature of the signed token;
determine that the signed token is assigned to the medical device;
determine that the signed token has not expired;
determine that the signed token has not been previously received with any other certificate signing request;
obtain a certificate from a certificate authority using the certificate signing request and the signed token; and
send the certificate to the medical device.

2. The system of claim 1, further comprising a setup device configured to:
obtain, from the verification system, a second public key and a second secret key associated with the setup device;
establish a near-field wireless communication connection to the medical device;
generate the identity data;
generate the signed token using the identity data and the second secret key; and
send the signed token to the medical device.

3. The system of claim 2, wherein the setup device is further configured to send network communication data to the medical device, wherein the network communication data comprises data for connecting to a local area network.

4. The system of claim 1, wherein the verification system is further configured to:
analyze the signed token to determine an expiration criterion, wherein to determine that the signed token has not expired, the verification system is configured to evaluate the expiration criterion; and
store usage data representing receipt of the signed token in connection with the certificate signing request.

5. The system of claim 1, wherein the verification system is further configured to:
obtain a certificate revocation list from the certificate authority; and
send the certificate revocation list to a plurality of medical devices.

6. The system of claim 1, wherein the verification system receives the certificate signing request from the medical device via a local area network of a hospital, and wherein to obtain the certificate from the certificate authority, the verification system is further configured to send the certificate signing request and the signed token to the certificate authority hosted in a cloud provider network separate from the local area network.

7. An infusion pump comprising:
a secure data store;
a processor configured to:
obtain, from a setup device, a signed token comprising a device identifier uniquely assigned to the infusion pump;
generate, using the device identifier, a secret key and a corresponding public key;
store the secret key in the secure data store;
generate a certificate signing request comprising the public key;
send the certificate signing request and the signed token to a verification system, wherein the verification system is separate from the setup device;
receive a certificate from the verification system;
establish, using the certificate, a secure network connection to a device; and
receive, via the secure network connection, data regarding a medication; and
a motor controller configured to cause the medication to be administered from a medication container.

8. The infusion pump of claim 7, wherein to establish the secure network connection to the device, the processor is further configured to establish the secure network connection to a hospital medication safety system.

9. The infusion pump of claim 7, wherein the processor is further configured to obtain, from the setup device, network configuration information, wherein the secure network connection is established using the network configuration information.

10. The infusion pump of claim 7, wherein the processor is further configured to:
prefetch a certificate revocation list from the verification system;
receive a second certificate from the device during establishment of the secure network connection; and
determine, using the certificate revocation list, that the second certificate has not been revoked.

11. The infusion pump of claim 7, wherein the processor is further configured to:
prefetch a certificate revocation list from the verification system;
receive, from a hospital medication safety system, a drug library and a second certificate;
determine, using the certificate revocation list, that the second certificate has not been revoked; and
verify a signature of the drug library using a second public key associated with the second certificate.

12. The infusion pump of claim 7, wherein the processor is further configured to:
determine to generate a new secret key and a corresponding new public key to replace the secret key and the public key;
generate the new secret key and the new public key;
store the new secret key in the secure data store;
delete the secret key from the secure data store;
generate a second certificate signing request comprising the new public key;
establish, using the certificate, a second secure connection to the verification system;
send the second certificate signing request to the verification system; and
receive a second certificate from the verification system in response to the second certificate signing request.

13. A computer-implemented method comprising:
as performed by a verification system comprising one or more processors configured to execute specific instructions,
providing a secret key to a setup device, wherein the secret key is associated with a public key;
receiving, from a medical device, a certificate signing request and a signed token;
verifying a signature of the signed token using the public key;
determining that the signed token is uniquely assigned to the medical device and the certificate signing request is for the medical device;
determining that the signed token has not expired;
determining that the signed token has not been used with any prior certificate signing request; and
obtaining a certificate from a certificate authority on behalf of the medical device.

14. The computer-implemented method of claim 13, further comprising extracting expiration data from the signed token, wherein determining that the signed token has not expired is based on the expiration data.

15. The computer-implemented method of claim 13, further comprising extracting device identifier data from the signed token, wherein determining that the signed token is uniquely assigned to the medical device is based on the device identifier data.

16. The computer-implemented method of claim 13, wherein obtaining the certificate comprises sending the certificate signing request and the signed token to the certificate authority.

17. The computer-implemented method of claim 13, further comprising:
pre-fetching a certificate revocation list from the certificate authority;
caching the certificate revocation list; and
sending the certificate revocation list to the medical device.

18. The computer-implemented method of claim 13, further comprising:
establishing a secure connection with the medical device based at least partly on the certificate;
receiving, from the medical device via the secure connection, a second certificate signing request;
determining, based at least partly on a device identifier in the second certificate signing request matching a device identifier in the certificate used to establish the secure connection, that the second certificate signing request is for the medical device; and
obtaining a second certificate from the certificate authority on behalf of the medical device.
